(19) United States
(12) Patent Application Publication
(10) Pub. No.: US 2006/0084186 A1
(43) Pub. Date: Apr. 20, 2006

(54) System and method for identifying proteins

(76) Inventors: Alison Chaiken, Fremont, CA (US); Manish Sharma, Sunnyvale, CA (US); Alex Bratkovski, Mountain View, CA (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 10/969,439

(22) Filed: Oct. 19, 2004

(51) Int. Cl. G06F 19/00; G01N 33/543

(52) U.S. Cl. 436/518; 702/19

(57) ABSTRACT

A method is disclosed for identifying one or more specific proteins in a heterogeneous test solution with a plurality of micro-cantilevers which have two or more conductive arms coupled to an end area having a higher resistance than the conductive arms. The method includes the operation of heating each end area of the plurality of micro-cantilevers by passing a current through each end area. A further operation includes increasing a temperature of the heterogeneous test solution using each heated end area of each of the plurality of micro-cantilevers. Another operation involves measuring a temperature change in the heterogeneous test solution that occurs when a specific protein denatures while heating the heterogeneous test solution with the plurality of micro-cantilevers. A further operation includes identifying the one or more specific proteins in the heterogeneous test solution according to the temperature at which each of the specific proteins denatured.

### FIELD OF THE INVENTION

The present invention relates generally to the field of protein identification.

### BACKGROUND

The completion of mapping of the genetic code for humans has ushered in a new frontier for science. Researchers are now undertaking inquiries to understand the underlying proteins which make up the genetic code. Proteomics is the study of the proteins which make up the 23 amino acids that can be created from the four nucleic acid bases of genomics. Proteomics can be much more complex than genomics due to the extremely large number of proteins that can be “spelled” by the 23 amino acids. Further, proteomic assays are usually quantitative, providing information about the concentration, physical, chemical, and biological characteristics of particular proteins, not just their presence or absence.

Two common macroscopic methods for purifying proteins are currently used. First, gel electrophoresis is a technique that involves electric-field induced migration of proteins through gels. Electrophoresis can be inexpensive and quite sensitive. However, it is slow since separation is based on diffusion of the proteins through a gel medium. A second common method for protein identification is matrix-assisted laser desorption ionization (MALDI). MALDI is a mass spectroscopic, time-of-flight method based on laser desorption of bio-molecules in a vacuum chamber. While sensitive, specific (due to many fragments of different masses from a given protein) and potentially quantitative, MALDI is slow and requires expensive, non-portable equipment.

Many new proteomic screening methods based on antibody-antigen binding have been developed using lithographic technology to design assays built into microchips. The microchips can contain devices designed to be coated with complementary antibodies which allow a specific protein to bind to the device. These new assays have the advantage of being relatively fast and inexpensive, but they are not very general due to their reliance on the availability of complementary antibodies. When complementary antibodies are not available, non-specific binding to the device may occur, but then no means of identification is possible.

Existing micro-cantilever based on-chip assays rely on optical detection of static cantilever deflection when a protein binds to it and unfolds or otherwise changes shape. A laser, LED, or other optical source can be used to measure the amount of deflection, which can be used to determine specific types of proteins. When complex biological fluids like blood are used rather than test solutions like buffered saline, optical opacity may be a serious problem. This problem could be addressed by using a more sophisticated measurement technique that is insensitive to analyte opacity.

On-chip detection, identification and quantification of proteins in complex solutions such as blood are highly desirable for a large number of health monitoring and screening applications. For example, rapid home tests for HIV infection or pathogens in food could save countless lives. On-chip detection methods can be faster and cheaper than those involving high-vacuum systems or lasers. However, most on-chip tests identify proteins through observation of specific antigen-antibody binding reactions. When on-chip detectors utilize non-specific binding mechanisms, they lose the ability to identify particular proteins. An assay is highly desirable in which binding does not depend on the availability of an antibody to each of the proteins of interest, but is capable of distinctly identifying individual proteins.

### SUMMARY OF THE INVENTION

A method is disclosed for identifying one or more specific proteins in a heterogeneous test solution with a plurality of micro-cantilevers which have two or more conductive arms coupled to an end area having a higher resistance than the conductive arms. The method includes the operation of heating each end area of the plurality of micro-cantilevers by passing a current through each end area. A further operation includes increasing a temperature of the heterogeneous test solution using each heated end area of each of the plurality of micro-cantilevers. Another operation involves measuring a temperature change in the heterogeneous test solution that occurs when a specific protein denatures while heating the heterogeneous test solution with the plurality of micro-cantilevers. A further operation includes identifying the one or more specific proteins in the heterogeneous test solution according to the temperature at which each of the specific proteins denatured.

### BRIEF DESCRIPTION OF THE DRAWINGS

FIG. 1 is a block diagram of a proteomic identification system in accordance with an embodiment of the present invention;

FIG. 2 depicts an embodiment of a micro-cantilever having a heated end;

FIG. 3a is a diagram showing a cross sectional view of a proteomic test chip in accordance with an embodiment of the present invention;

FIG. 3b is a diagram of a front view of a proteomic test chip in accordance with an embodiment of the present invention; and

FIG. 4 is a flow chart depicting an embodiment of a method for identifying one or more specific proteins in a heterogeneous test solution.

### DETAILED DESCRIPTION

Reference will now be made to the exemplary embodiments illustrated in the drawings, and specific language will be used herein to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention.

Calorimetry can be defined as the measurement of the amount of heat evolved or absorbed in a chemical reaction, change of state, or formation of a solution. A calorimetric method of protein identification need not rely on specific binding through the use of antibodies, which must be specifically produced for each protein. Calorimetry can be used to identify specific proteins based upon the temperature at which the specific proteins denature. Denaturing occurs when a protein's structure unfolds in response to a stimulus such as heat, alkali, or acid. Different proteins can denature at specific temperatures.

The process of denaturing is usually an endothermic process, absorbing heat from the surrounding liquid. However, exothermic processes, giving off heat to the surrounding liquid, can also be measured. Whether the process is endothermic or exothermic depends upon the specific protein and the type of bonds creating the protein structure. The process of denaturing can result in a change in enthalpy of a test sample. The change can be either positive or negative, depending upon the specific protein which denatured.

An index of the transition temperatures at which different proteins denature can be created. The specific types of proteins in a test sample can then be determined by referencing changes in enthalpy of the test sample at specific temperatures defined by the index. A variety of proteins present in complex heterogeneous test solutions, such as blood or other biological fluids, can then be discovered according to protein transition temperatures detected as the test solution is heated. Measuring changes in specific heat or latent heat produced when a protein denatures can require expensive equipment and elaborate setups, however. Moreover, relatively large volumes of fluid may be needed for successful measurements. The present invention provides a method for quickly, inexpensively, and accurately detecting protein temperature transitions in biological samples.

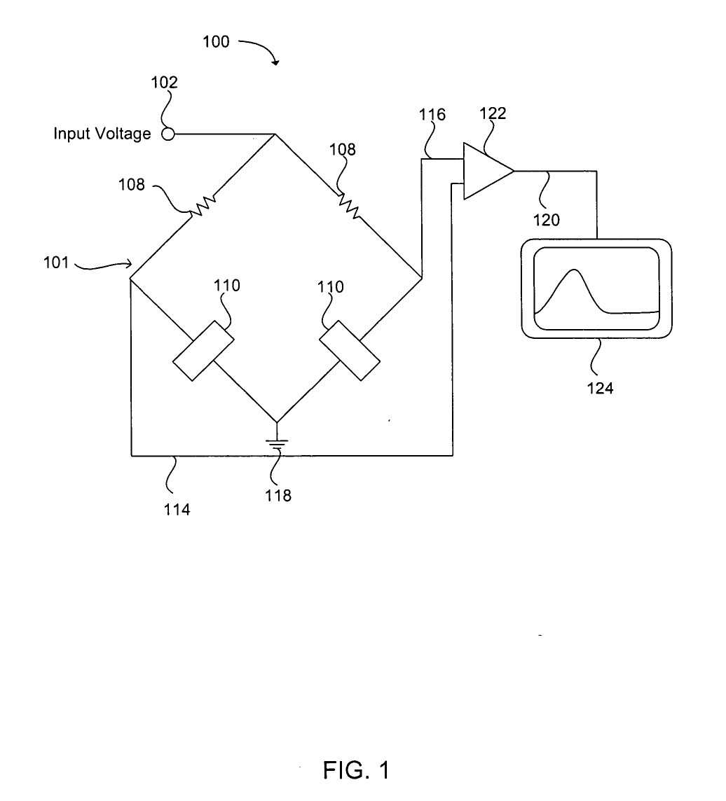

In order to address the problems described and to efficiently, accurately, and relatively inexpensively detect protein temperature transitions, the present invention provides a system and method for identifying one or more specific proteins in a heterogeneous test solution with a plurality of micro-cantilevers that have conductive arms coupled to ends having a higher resistance than the arms, as illustrated in the example embodiment of FIG. 1. The proteomic identification system 100 shown in this example can be comprised of micro-cantilevers arranged in a Wheatstone bridge configuration 101.

Due to their sensitivity, Wheatstone bridge circuits are advantageous for the measurement of resistance, inductance, and capacitance. A Wheatstone bridge circuit configuration consists of four resistive elements electrically coupled in a diamond orientation 101. An input voltage 102 is applied between the input 102 and ground 118 of the diamond and the output voltage 120 is measured between two measurement taps 114 and 116. When the output voltage, the potential difference between 114 and 116, is zero, the bridge is said to be balanced. One or more of the legs of the bridge may be a resistive element, such as a doped micro-cantilever 110 in this example. The other legs of the bridge are simply completion resistors 108 with resistances set to be substantially equal to that of the cantilever(s) when the bridge is balanced. The completion resistors 108 may also be potentiometers, enabling each completion resistor to be adjusted to allow the bridge to be balanced.

As the resistance of one of the legs changes, due to a change in temperature of a doped micro-cantilever, for example, the previously balanced bridge can become unbalanced. This unbalance can cause a difference in potential to appear across the middle of the bridge. This difference may be measured using a lock-in amplifier 122. The output 120 of the lock-in amplifier can be sent to a recording device 124 and the change in potential can be recorded over time.

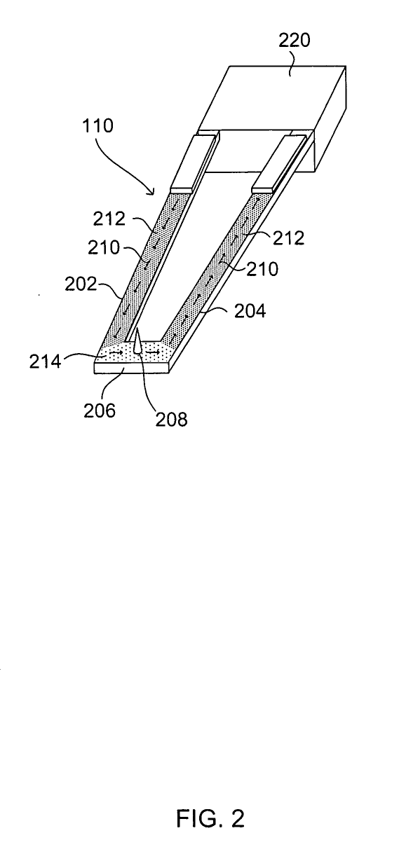

A micro-cantilever and end 110 can be used both to heat a test solution and to accurately measure any changes in enthalpy in the test solution, as shown in FIG. 2. The cantilever and end 110 can be formed from semiconductor material through a series of known etching processes. In one embodiment, each cantilever can be formed from single crystal silicon. Additional processes can enable the cantilevers to be used as heaters, as will be discussed. A cantilever configured to be used as a heater can be comprised of a first arm 202 connected to a substrate area 220. A tip 208 can be connected to an end area 206. The end area can be connected to the first arm 202 and a second arm 204. The second arm 204 can be connected between the end area 206 and the substrate area 220. A typical cantilever can be about 15 microns long with about 1 micron wide arms. However, cantilevers can be formed having a wide range of sizes, the size being limited only by limits in lithography. Current limits create a minimum dimension of about 0.1 microns, but future improvements in lithography and MEMS construction may further reduce the size of the cantilevers. The cantilever 110 can be used to make extremely accurate measurements of changes in temperature in small amounts of liquid. Each cantilever can have a size on the order of a few square microns up to tens of square microns.

A plurality of cantilevers can be incorporated on a substrate such that their characteristics are tuned over a range of responses. For example, the cantilevers can be made of different sizes and the amount of doping in the arms of the cantilevers can be changed so they have different time constants. Using an array of cantilevers placed on the same substrate of a chip can enable measurements of varying sensitivities which can be done at the same time.

The heat capacity of a substance is the amount of heat required to change its temperature by one degree. Due to its extremely small mass, the cantilever can have a very small heat capacity which can enable small changes in temperature to be measured within a span of a few microseconds. In one embodiment, a typical measurement can be done in 5 to 20 microseconds. A shorter time span can be accomplished by further reducing the size of the cantilever. The rapid measurement of changes in temperature greatly increases the accuracy of temperature measurements and allows for a substantially adiabatic system, where the influences of the container can be essentially negligible. Thus, the cantilever's extremely small size, combined with a sensitive temperature detection system, enable the invention to have the temperature accuracy necessary to detect changes in enthalpy in small test solutions which occur when specific proteins denature.

The cantilever 110 can be configured to act as a heating device by creating heavily doped areas 212 on the first and second arms 202 and 204. The heavily doped areas 212 can allow the arms to be electrically conductive. A lighter doped area 214 can be formed along the end area 206 and the tip 208. The lighter doped area 214 can have a higher resistance than the heavily doped areas 212. The higher resistance can be used to increase the temperature of the lighter doped area 214. For example, a voltage, or potential difference, can be applied between the first and second arms, inducing a current flow 210 along the first arm 202, past the tip 208, and back through the second arm 204. The higher resistance in the lighter doped area 214 and tip 208 can cause the lighter doped area 214 to heat up. The heat can be transferred to a test solution through the end area 206.

A change in temperature in the test solution, due to a chemical reaction or denaturing proteins, can be detected by the cantilever 110. The change in temperature can alter the resistance of the cantilever. A small change in resistance in a cantilever 110 can cause the Wheatstone bridge 101 to become unbalanced, causing a voltage to appear across the middle of the bridge. This voltage difference can induce a current which can be accurately detected by a lock-in amplifier, as previously discussed.

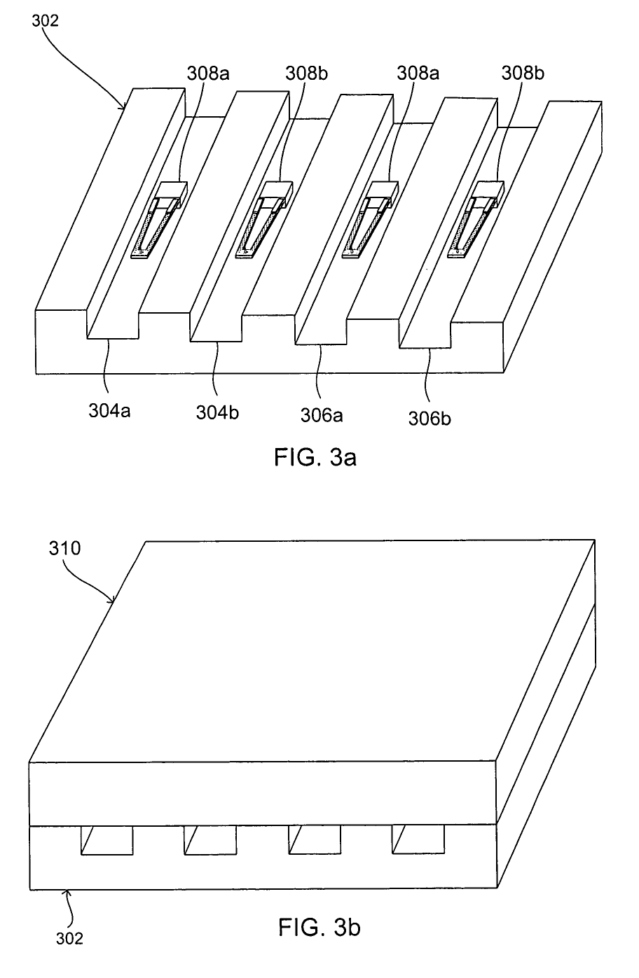

One embodiment of the proteomic identification system comprises a plurality of micro-cantilevers 110 configured in an array of Wheatstone bridges 101 (FIG. 1). Each Wheatstone bridge can be configured to have two heated cantilevers 110 (FIG. 1), a control cantilever 308a and a test cantilever 308b, as shown in FIG. 3a. The cantilevers 308a and 308b can be located in pairs of microfluidic channels comprising control channels 304a and 306a, and test channels 304b and 306b in a proteomic test chip 302. Each microfluidic channel can have a width of 10 to 500 microns. A typical microfluidic channel may be 100 microns wide. Each of the cantilevers 308a and 308b can be functionalized with a non-specific binding agent for proteins. The non-specific binding agent can be comprised of non-fat dried milk, bovine serum albumin, or any other protein configured to act as a non-specific binding agent for proteins in a test solution. A buffered solution can be injected through each control channel 304a and 306a. The buffered solution can have similar characteristics to the test solution. The similar characteristics can include having a similar temperature, pH, and salt concentration. The similar characteristics should allow the buffered solution to have substantially similar thermal properties as the test solution.

Each Wheatstone bridge circuit 101 (FIG. 1) can be balanced at room temperature, or a series of temperatures, by adjusting the resistance of the completion resistors. The completion resistors can be manually or electrically adjusted. A computer can be used to adjust the completion resistors to a predetermined value for a given temperature. The computer can be external to the proteomic identification system 100. Alternatively, the computer can comprise a microprocessor located on-chip and in communication with the proteomic identification system 100.

An unknown test solution can be injected into the test channels 304b and 306b. The control cantilevers 308a in the control channels and test cantilevers 308b in the test channels can then be heated, allowing the fluid surrounding the cantilever ends to be heated. The test and control channels can enable more accurate temperature data to be obtained, as most external influences such as vibration or changes in room temperature, will occur substantially equally to the fluids in both channels. Thus, most external effects can be negated by measuring the difference between the fluids in the test channel and control channel. Each test channel 304b and 306b can have a plurality of associated control channels to increase the accuracy of each test. Alternatively, a plurality of test channels can all use the same control channel. A separate test can be completed in parallel in the control and test channels 306a and 306b, respectively. Further, parallel channels can be incorporated on the same chip, allowing multiple tests to be performed. Alternatively, a plurality of the same test can be performed at one time, increasing the statistical accuracy of the test's results.

In another embodiment, the proteomic test chip 302 can comprise a plurality of microfluidic channels. A cantilever substrate 310 comprising a plurality of cantilevers can be configured so that the substrate 310 can be placed on top of the test chip 302 in a manner allowing one or more cantilevers 110 to be located in each microfluidic channel, as shown in FIG. 3b.

A range of methods may be used to heat the end area of each cantilever, depending upon the proteins expected to be found in the test solution and the required sensitivity needed to detect changes in temperature which occur when specific proteins denature in the test solution. One method for heating the cantilever end areas involves causing a pulse of current, having a predetermined amplitude and length, to flow across the cantilever end areas. This can be accomplished by pulsing a voltage on and off between the input 102 and ground 118 of each bridge circuit 101 (FIG. 1).

In one embodiment, a potential difference of one volt between the input 102 and ground 118 of each Wheatstone bridge 101 can be pulsed on and off (FIG. 1). The total amount of resistance contributed by each resistive device in the Wheatstone bridge will determine the amplitude of the current pulse. The resistance of the cantilevers can be determined by the amount of doping injected into the arms 202, 204 and end area 206 (FIG. 2). The amplitude of the current pulse can be increased by increasing the voltage across the Wheatstone bridge 101 or using cantilevers 110 and completion resistors 108 having a lower resistance (FIG. 1).

The actual voltage can vary between a few millivolts and tens of volts, depeding upon the resistance and required current amplitude. The amount of current used can be related to the amount of heat desired to be transferred to each cantilever end area and the test and buffer solutions in which they are located. In one embodiment, a current pulse can be sent through the cantilevers having a length of 100 microseconds. The desired length of the current pulse can depend upon the length of time it may take for a protein to denature. The length of the current pulse and thus the highest temperature in the experiment can be dependent upon the specific type of protein being analyzed. A shorter current pulse can also enable more accurate temperature testing, which will be discussed further below.

The pulse can be sent to the control cantilever 308a located in the buffer solution in the control channel 304a and also to the test cantilever 308b located in the test solution in the test channel 304b. If no chemical reactions take place, the change in temperature in the buffer solution and the test solution should be substantially equal, permitting the Wheatstone bridge circuit 101 (FIG. 1) to remain substantially balanced. However, if a protein denatures, an endothermic reaction can occur. This reaction causes heat to move from the cantilever to the protein which reduces the temperature of the test cantilever 308b. It is also possible for an exothermic reaction to occur which can cause heat to move from the solution to the test cantilever 308b and increase the temperature of the test cantilever 308b. A change in temperature will cause a change in the resistance of the test cantilever, causing the bridge circuit to become unbalanced and resulting in a voltage measured across the middle of the circuit. The resulting voltage can be proportional to the difference in temperature between the control cantilever 308a and the test cantilever 308b.

Using pulsed current to heat the cantilevers can be advantageous. In pure water, the heat from the cantilever end area 206 travels through the test solution at a rate of 0.4 micrometers per microsecond. A change in heat can be measured in 5 to 20 microseconds. In 20 microseconds the heat would only travel about 8 micrometers. Thus, if the cantilever end 208 is located more than 8 micrometers away from the channel walls, the short pulses of current can allow the system to be quasi-adiabatic, since parasitic heat leaks to the channel walls may be slower than the phase transition and thermal transients of interest, allowing for more accurate testing. Performing measurements during the short pulse period, however, can be challenging.

Alternatively, the cantilever end can be heated by applying a DC ramp current through the cantilevers 110. A DC ramp current can be injected into each cantilever in a bridge circuit 101 by applying a small DC voltage between the top and bottom of the bridge circuit 101. The DC voltage can then be ramped up over time to a predetermined maximum. In one embodiment, the DC ramp can go from 0 volts DC to 1 volt DC over a one second time frame. The increasing DC current resulting from the increasing voltage can raise the temperature of the control and test solutions, as previously discussed. Any protein denaturing which occurs during the ramp-up can result in a difference in temperature between the test and control cantilevers, causing the resistance of the cantilevers to change, which will unbalance the Wheatstone bridge circuit 101 and result in a voltage 120.

Accurately measuring small changes in DC current can be difficult. Lock-in detection using a lock-in amplifier can be used to increase the accuracy of the measurement. Lock-in detection can be used to perform a quasi-DC measurement at a non-zero frequency with a smaller bandwidth than DC. The smaller bandwidth can reduce the amount of noise on the signal. In one embodiment, lock-in detection can be performed by adding a small AC signal, such as a 4 millivolt, 35 kHz signal, to the DC ramp or pulse current input. The output can be modulated (multiplied) by the small AC signal at the input. The modulation will produce sidebands corresponding to the sum and difference of the input signal. As the input signal to be measured and the reference signal are of the same frequency, the difference frequency is essentially zero and the output is a DC signal which is proportional to the amplitude of the input signal. Noise present in the lock-in detection signal can be filtered using a low pass filter. The change in current of the DC ramp will be proportional to the change in the lock-in detection signal current since both depend linearly on the temperature-dependent resistance of the cantilever. Using a lock-in amplifier device, full scale sensitivities of 0.1 pA can be realized.

Another embodiment of the invention can involve performing differential measurements with the two cantilevers in the Wheatstone bridge without heating the cantilevers. In this embodiment, the lock-in amplifier device can be used to measure changes in temperature of the test solution caused by external sources. External sources can include chemicals designed to cause chemical reactions or external heat sources such as a laser. The external sources can be added to the test solution, the control solution or both. This embodiment can also be used to determine a quantity of a reactant in the test solution. For example, it can be assumed that a reactant A exists in a test solution. A different reactant B, which will react with A in an exothermic reaction to form AB, can be added to the test solution. The production of AB can be thermally monitored as reactant B is added to the test solution. When heat production in the test solution stops, it can be determined that all of reactant A in the test solution has been used to produce AB. The original amount of reactant A in the test solution can be determined by the amount of reactant B it took to use up all of the reactant A.

The Wheatstone bridge circuit 101 configuration can be used to measure the heat capacity in a test solution. Heat capacity C is the derivative of energy E with respect to temperature T, leading to the equation:

$$C = \frac{ⅆ E}{ⅆ T}$$

It has been found that resistance R is linear with respect to temperature T. Power can be input for a time τ. The change in resistance ΔR of the cantilevers in the bridge circuit can be determined by using the lock-in detector to measure the change in current, and determining the resistance with Ohm's law,

$$Δ R = \frac{V}{Δ I} ,$$

where V is the DC voltage applied between the top and bottom of the bridge circuit. Alpha (α) can be defined as the temperature coefficient of resistivity, with α=dR/dT, the change in resistance per change in temperature. Change in heat capacity, ΔC, can then be defined as:

$$Δ C = \frac{P \times τ \times α}{Δ R}$$

In an example embodiment, the cantilever can have a resistance of about 1600 ohms. Two leads can be connected to each cantilever to measure current and voltage. Alternatively, four leads can be connected to each cantilever, with two leads used to measure current and two leads used to measure voltage. Using four leads, the effects of the leads in the measurement can be minimized. A typical value of dR/dT for a cantilever can be 5 ΩK, or 5 ohms per degree Kelvin. A Wheatstone bridge having two matched resistors and two cantilevers, each with a resistance of 1600 Ohms, can have a resistance of 3,200 Ohms across the bridge. A change in resistance ΔR of 0.2 ohms can be measured using the bridge. The sensitivity of the system to measure a change in temperature can be determined according to the equation ΔT=ΔR/(dR/dT). Thus, the system can have a sensitivity of measuring a change in temperature of 0.2K/(5 Ω/K)=40 mK. A change in energy can be found by multiplying the change in temperature by the heat capacity C of the cantilever. The heat capacity is dependent upon the material from which the cantilever is made. In one embodiment, the heat capacity of a micromachined cantilever formed from silicon is 50 picojoules/K. Thus, the overall energy resolution can be:

$$Δ E = Δ T \times C = 40 \times {10}^{- 3} K * 50 \times {10}^{- 12} \frac{J}{K} = 2 p J$$

One can use different materials for cantilevers or their parts responsible for having a large temperature coefficient of resistivity dR/dT. For example, some conducting oxides like La₁₋ₓCaₓMnO3 or Ba₍₁₋ₓ₎SrₓO, which have large dR/dT, can enable the dR/dT for cantilever end areas to be increased.

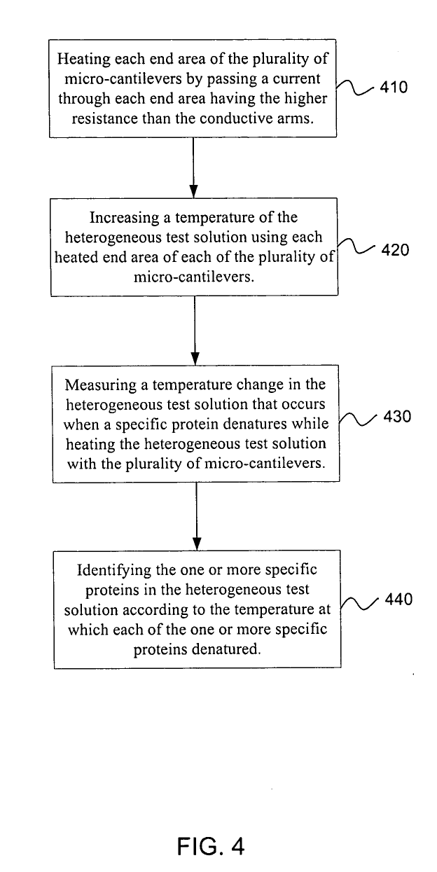

Another aspect of the invention provides a method for identifying one or more specific proteins in a heterogeneous test solution with a plurality of micro-cantilevers having conductive arms coupled to an end area 206 (FIG. 2) having a higher resistance, as depicted in the flow chart of FIG. 4. The method includes the operation of heating each end area of the plurality of micro-cantilevers by passing a current through each end area having the higher resistance than the conductive arms, as shown in block 410. The current can follow a path through one of the conductive arms on each cantilever, through the resistive end area of the cantilever, and out through another conductive arm. The current can be sent in short pulses. The pulses can have durations of less than one millisecond. In one embodiment, the pulses can be created by applying a voltage difference of one volt between the arms of each cantilever for the duration of the pulse. Alternatively, the current can be sent through the cantilevers in a DC ramp. The DC ramp can be created by applying a voltage difference between each cantilever's arms. In one embodiment, the voltage can be ramped between zero volts and one volt.

A further operation includes increasing a temperature of the heterogeneous test solution using each heated end area of each of the plurality of micro-cantilevers, as shown in block 420. The voltage between the arms of the cantilevers can be applied with an amplitude and for a duration of length sufficient to heat the test solution to a temperature which can cause the one or more specific proteins to denature. This temperature may be between 40° C. and 100° C.

Another operation involves measuring a temperature change in the heterogeneous test solution that occurs when a specific protein denatures while heating the heterogeneous test solution with the plurality of micro-cantilevers, as shown in block 430. The change in temperature of the temperature in the heterogeneous test solution can be accurately measured by configuring one or more of the cantilevers in a Wheatstone bridge configuration. In one embodiment, differential scanning can be performed by forming a Wheatstone bridge comprising two cantilevers with heated ends and two completion resistors, all having a substantially equal resistance. The heated end of a first cantilever, or test cantilever, can be placed in a test solution. The heated end of the second cantilever, or control cantilever, can be placed in a control solution. Any external changes in environment should affect both the test and control solutions equally and cause them to both have substantially the same temperature. A temperature change in the test solution that is caused by protein denaturation can cause the temperature of one of the test cantilevers to change, which can change the resistance of the test cantilever and unbalance the Wheatstone bridge. When the Wheatstone bridge becomes unbalanced, a potential across the bridge can be created, causing a current to flow.

The current flowing through the unbalanced bridge can be accurately measured using a lock-in amplifier. A small AC signal can be applied across the Wheatstone bridge. The lock-in amplifier can then be used to accurately detect the AC current. The amount of current flowing through the Wheatstone bridge can be proportional to the current flow caused by the change in resistance of the test cantilever which can unbalance the Wheatstone bridge. The AC signal frequency and amplitude can be set so that it will not heat the test solution. In one embodiment, it can be set to an amplitude of 4 millivolts at a frequency of 35 kHz. The current flow through the Wheatstone bridge can be proportional to the change in resistance, which is relative to the change in heat capacity of the test solution.

A further operation involves identifying the one or more specific proteins in the heterogeneous test solution according to the temperature at which each of the one or more specific proteins denatured, as shown in block 440. By measuring the change in heat capacity of the test solution compared to the control solution as the temperature is scanned, it can be determined when a protein denatures and at approximately what temperatures the denaturation took place. Each protein can denature at a temperature defined by the physical makeup of the protein and the types of chemical bonds holding it together. Thus, one or more specific proteins can be identified in the heterogeneous test solution by determining at what temperature the proteins denatured.

The proteomic identification system can be configured as an inexpensive consumable part or miniature integrated system having a library of heat capacitances stored within the system in a digital memory device representing the temperature at which a plurality of different proteins denature. The digital memory device can be comprised of any device capable of storing a sufficient quantity of digital information. For example, the digital storage device can be comprised of random access memory (RAM), magnetic RAM, flash memory, a magnetic hard drive, an optical disk, or a combination of these types of memory and storage. The system can further comprise a microprocessor, energy source, and a wireless communication device that can be embedded in living tissue for constant monitoring. The microprocessor can be a digital signal processor, a microcontroller, or a reconfigurable field programmable gate array. The wireless communication device can be configured to operate according to an IEEE wireless standard including IEEE 802.11, IEEE 802.15, and IEEE 802.16. The IEEE wireless standards can include WiFi, ultrawideband, Bluetooth®, mesh networking, and Zigbee®. The wireless communication device can be any device capable of communicating to an external device. For example, the proteomic identification system can be implanted into a mouse which can then be used to remotely detect the presence of chemical agents in a hazardous area.

A large number of proteins typically occur in nature. In order to organize the proteins, they are typically arranged by placing them into classifications of families comprising related proteins. The families can consist of similar proteins that have similar structures arising from similar peptide chains and bonds between chains (which cause folding). As a protein denatures, the temperature of the fluid can be measured using the proteomic identification device and recorded over time to produce a calorimetric graph which can serve as the proteins characteristic signature profile. Similar proteins belonging to the same family can produce similar signatures. Two added benefits of the proteomic identification device are that, first, proteins can be identified which are related to each other; and second, a new protein can be identified which is not in the library of heat capacitances (ie hadn't been previously measured) by relating its signature to existing ones in the database.

Some different proteins can denature at substantially similar temperatures. These proteins may even have similar characteristic signature profiles from their calorimetric graph. However, these proteins may react differently to other environmental changes. In order to increase the specificity of protein identification, the denaturation temperature dependence on salinity or pH can be measured by controlling the composition of a test solution. Proteins with similar denaturation temperatures and heats of denaturation may have a dissimilar dependence of these properties on salinity and pH.

The proteomic identification system can be useful in performing microcalorimetric measurements to determine one or more proteins in a heterogeneous test solution. Using micro-cantilevers having an extremely small size and weight, quasi-adiabatic measurements can be performed, allowing measurements with sensitivities of a few nJ/K to be detected. A high resolution of the measurements can be achieved by a smart statistical processing of the data coming from an array of sensors. Such sensitivity can allow the system to rapidly detect protein in a solution having less than 1 micromolar concentration in the heterogeneous test solution. The system can be configured to be relatively inexpensive and can analyze test samples almost instantaneously. The proteomic identification system fulfills a long sought need for a system capable of inexpensively, quickly, and accurately detecting a plurality of non-specific proteins in a heterogeneous solution.

It is to be understood that the above-referenced arrangements are illustrative of the application for the principles of the present invention. It will be apparent to those of ordinary skill in the art that numerous modifications can be made without departing from the principles and concepts of the invention as set forth in the claims.

What is claimed is:

1. A method for identifying one or more specific proteins in a heterogeneous test solution using a plurality of micro-cantilevers having conductive arms coupled to an end area having a higher resistance than the conductive arms, comprising:
heating each end area of the plurality of micro-cantilevers by passing a current through each end area having the higher resistance than the conductive arms;
increasing a temperature of the heterogeneous test solution using each heated end area of each of the plurality of micro-cantilevers;
measuring a temperature change in the heterogeneous test solution that occurs when a specific protein denatures while heating the heterogeneous test solution with the plurality of micro-cantilevers; and
identifying the one or more specific proteins in the heterogeneous test solution according to the temperature at which each of the one or more specific proteins denatured.

2. A method as in claim 1, further comprising configuring the plurality of micro-cantilevers as single crystal micro-cantilevers having sharpened tips.

3. A method as in claim 1, further comprising heavily doping the conductive arms of the plurality of micro-cantilevers to enable the arms to be conductive.

4. A method as in claim 3, further comprising increasing the temperature coefficient of resistivity of the plurality of micro-cantilevers by doping one or more end areas of the plurality of micro-cantilevers with one or more conducting oxide thermistor materials selected from the group consisting of La₁₋ₓCaₓMnO3 and Ba₍₁₋ₓ₎SrₓO, where x is an integer.

5. A method as in claim 3, further comprising doping each end area of the cantilevers less than the conductive arms to enable the each end area to have the higher resistance than the conductive arms.

6. A method as in claim 1, further comprising configuring the plurality of micro-cantilevers in a Wheatstone bridge circuit configuration having two or more micro-cantilevers with heatable end areas.

7. A method as in claim 6, further comprising configuring the plurality of micro-cantilevers in the Wheatstone bridge circuit wherein the Wheatstone bridge circuit comprises two resistors and two micro-cantilevers, the cantilevers each having a heatable end area.

8. A method as in claim 6, further comprising positioning at least one of the heatable end areas of the two or more micro-cantilevers in a micro-fluidic cell containing a test solution.

9. A method as in claim 6, further comprising positioning each of the heatable end areas of the two or more micro-cantilevers in a micro-fluidic channel.

10. A method as in claim 9, further comprising placing a heatable end area of a first cantilever in a first micro-fluidic channel wherein a buffer solution is located in the first micro-fluidic channel.

11. A method as in claim 9, further comprising placing a heatable end area of a second cantilever in a second micro-fluidic channel wherein a test solution is located in the second micro-fluidic channel.

12. A method as in claim 1, further comprising performing a measurement using a heatable end area of a cantilever in a micro-fluidic channel containing a test solution and a plurality of heatable end areas of a plurality of cantilevers in a plurality of micro-fluidic channels containing a buffer solution.

13. A method as in claim 1, further comprising performing a plurality of measurements using a heatable end area of a cantilever in a micro-fluidic channel containing a buffer solution and a plurality of heatable end areas of a plurality of micro-cantilevers in a plurality of microfluidic channels containing a plurality of test solutions.

14. A method as in claim 7, further comprising balancing the Wheatstone bridge circuit by adjusting the resistance of each of the two resistors and each of the two micro-cantilevers with heatable end areas to be substantially equal.

15. A method as in claim 1, further comprising heating the end areas of the plurality of micro-cantilevers by sending the current through the end areas having the higher resistance, wherein the current is a pulse of electrical current.

16. A method as in claim 15, further comprising sending the pulse of electrical current through the end areas, wherein the pulse has a time duration sufficient to allow desired proteins in the heterogeneous test solution to denature in response to increased heat caused by the pulse of electrical current flowing through the end areas.

17. A method as in claim 1, further comprising heating the end areas of the plurality of micro-cantilevers with the current, wherein the current comprises a DC electrical current sent through the end areas.

18. A method as in claim 17, further comprising sending a small DC electrical current through the end areas and increasing DC electrical current until the end areas have reached a desired temperature for a predetermined amount of time.

19. A method as in claim 17, further comprising coupling a small AC electrical signal to the DC electrical current and using a lock-in amplifier to determine a change in AC current, wherein the change in AC current is proportional to the change in temperature of the end areas of the plurality of micro-cantilevers.

20. A method as in claim 1, further comprising applying a non-specific binding agent for proteins to one or more of the plurality of micro-cantilevers.

21. A method as in claim 20, further comprising exposing one or more of the plurality of micro-cantilevers in a Wheatstone bridge circuit to a buffered solution, the buffered solution having similar characteristics to a test solution.

22. A method as in claim 21, further comprising providing the buffered solution having similar characteristics to the test solution including temperature, pH, and salt concentration.

23. A method as in claim 22, further comprising changing the similar characteristics of the test solution when a first protein has a substantially similar temperature of denaturization as a second protein in order to distinguish between the first and second proteins.

24. A method as in claim 23, further comprising changing the similar characteristics of the test solution by altering one or more of the pH and salt concentration of the test and buffer solutions.

25. A method as in claim 1, further comprising determining an amount of a first reactant in the heterogeneous test solution by adding an amount of a second reactant configured to chemically react with the first reactant, wherein a heat of reaction can be measured using the plurality of micro-cantilevers and the amount of the first reactant can be determined by measuring the amount of the second reactant required for the reaction between the first reactant and the second reactant to stop when substantially all of the first reactant has reacted with the second reactant.

26. A proteomic identification system, comprising:
a test chip comprising a plurality of micro-fluidic channels;
a plurality of micro-cantilevers having conductive arms with end areas located near an end of the conductive arms, the end areas having a higher resistance than the conductive arms, wherein one or more of the end areas of the plurality of micro-cantilevers extend into one or more of the plurality of micro-fluidic device channels;
a current source configured to supply current to heat the end areas of the plurality of micro-cantilevers; and
a current measurement module configured to detect changes in current in the plurality of micro-cantilevers caused by a change in temperature of the micro-cantilever.

27. The proteomic identification system of claim 26, further comprising configuring one or more of the plurality of micro-cantilevers as a resistive device in a Wheatstone bridge configuration.

28. The proteomic identification system of claim 27, wherein the Wheatstone bridge configuration comprises two micro-cantilevers and two resistive devices.

29. The proteomic identification system of claim 27, wherein the two resistive devices are potentiometers configured to be adjusted to have substantially similar resistive properties as the two micro-cantilevers in order to balance the Wheatstone bridge circuit.

30. The proteomic identification system of claim 26, further comprising a digital memory device coupled to the test chip and configured to store heat capacities of a plurality of proteins.

31. The proteomic identification system of claim 30, further comprising a micro processing device in communication with the test chip and a solid state memory device and configured to compare changes in temperature of the end areas with the heat capacities of proteins stored on the digital memory device.

32. The proteomic identification system of claim 30, wherein the digital memory device is selected from a group consisting of random access memory (RAM), magnetic RAM, flash memory, a magnetic hard drive, and an optical disk.

33. The proteomic identification system of claim 26, further comprising a wireless communication system configured to enable remote access to the proteomic identification system.

34. The proteomic identification system of claim 33, wherein the wireless communication system is configured to operate according to a standard selected from a group of IEEE 802.11, IEEE 802.15, and IEEE 802.16.
